SEQUENCE LISTING (14 sequences)

SEQ ID NO: 1 - protein, 58 aa, Bos taurus [sequence omitted]

SEQ ID NO: 2 - protein, 24 aa, Homo sapiens
NGRRICLDLQAPLYKKIIKKLLES

SEQ ID NO: 3 - protein, 27 aa, Homo sapiens
GRELCLDPKENWVQRVVEKFLKRAENS

SEQ ID NO: 4 - protein, 34 aa, Homo sapiens
QIHFFFAKLNCRLYRKANKSSKLVSANRLFGDKS

SEQ ID NO: 5 - protein, 34 aa, Homo sapiens
ELRVRLASHLRKLRKRLLRDADDLQKRLAVYQAG

SEQ ID NO: 6 - protein, 12 aa, Homo sapiens
RRLRRMESESES

SEQ ID NO: 7 - protein, 21 aa, Homo sapiens
KRKKKGGKNGKNRRNRKKKNP

SEQ ID NO: 8 - protein, 379 aa, Homo sapiens
MASLPVLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLNDRARSLVVPAYAYRKLHPIQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 9 - protein, 424 aa, Homo sapiens
LAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRLECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPRSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPS

SEQ ID NO: 10 - protein, 5 aa, Artificial Sequence
  Synthetic construct
GGGGS

SEQ ID NO: 11 - DNA, 2742 bp, Actinomyces viscosus
  nanH gene for sialidase
atgacatcgcatagtcctttctcccggaggcgcctgccggccctcctgggctccctgccactggccgccaccggcctgatcgccgccgcacccccggcgcacgccgtccccacgtctgacggcctggccgacgtcaccatcacgcaggtgaacgcgcccgcggacggcctctactccgtcggcgatgtcatgaccttcaacatcaccctgaccaacaccagcggcgaggcccactcctacgccccggcctcgacgaacctgtccgggaacgtctccaagtgccggtggcgcaacgtcccggccgggacgaccaagaccgactgcaccggcctggccacgcacacggtgaccgccgaggacctcaaggccggtggcttcaccccgcagatcgcctacgaggtcaaggccgtggagtacgccgggaaggccctgagcaccccggagacgatcaagggcgcgacgagcccagtcaaggccaactcgctgcgggtcgagtcgatcacgccgtcgtcgagccaggagaactacaagctgggcgacaccgtcagctacacggtgcgcgtgcgctcggtgtcggacaagacgatcaacgtcgccgccaccgaatcctccttcgacgacctgggccgccagtgccactggggcggcctcaagccgggcaagggcgccgtctacaactgcaagccgctcacccacacgatcacgcaagccgacgtcgacgccggccgctggacgccatcgatcaccctgacggccaccggaaccgacggcgccaccctccagacgctcaccgccaccggcaacccgatcaacgtcgtcggcgaccacccgcaggccacgcccgcaccggcgcccgacgcgagcacggagctgccggcctcaatgagccaggcccagcacctggccgccaacacggccaccgacaactaccgcatcccggcgataccaccgcccccaatggggacctgctcatctcctacgacgagcgcccgaaggacaacggcaacggcggcagcgacgacccccaacccgaaccacatcgtccagcgccgctccaccgacggcggcaagacctggtcggcgcccacctacatccaccagggcacggagaccggcaagaaggtcggctactccgacccgagctacgtcgtcgatcaccagacgggcacgatcttcaacttccacgtcaagtcctacgaccagggctggggcggctcgcgcggcggcaccgacccggagaaccggggcatcatccaggccgaggtgtcgacctccacggacaacggctggacctggacgcaccgcacgatcaccgcggacatcacgaaggacaagccgtggaccgcgcgtttcgcggcctcgggccagggcatccagattcagcacgggccccacgccgggcgcctggtgcagcagtacacgatcaggaccgccggcgggccggtgcaggccgtctcggtctactccgacgaccacgggaagacgtggcaggccggcacgccgatcgggaccggcatggatgagaacaaggtcgttgagctctccgacggctccctcatgctcaactcgcgcgcctcggatggctccggcttccgcaaggtggcccactccaccgacggtgggcagacctggagcgagccggtgtccgacaagaacctgcccgactcggtggacaacgcccagatcatccgagccttcccgaacgccgcgccggacgacccgcgcgccaaggtgctgctgctgagccactcaccgaacccgcggccgtggtgccgtgaccgcggcaccatctcgatgtcctgcgacgacggcgcctcctggacgaccagcaaggtcttccacgagcccttcgtcggatacacgacgatcgcggtgcagtccgacggcagcatcgggctgctcagcgaggacgcccacaacggcgccgactacggcggcatctggtaccgcaacttcacgatgaactggctcggcgagcagtgcggccagaagccggcggagccgagcccgggccgtcgccgacggcggcaccctcagcggcaccgacggagaagccggccccgtcggccgcgccgagcgctgagcccacgcaggcaccggcaccatcctccgcgcccgagccgagcgctgcgcccgagccgagcaggccccggcgccggagcccacgaccgctccgagcacggagcccacaccggctcctgcgcccagtccgcacctgagcagaccgatgggccgaccgctgcgcccgcaccggagacgtcctctgcaccggccgccgaaccgacgcaggccccgacggtggcgccttctgttgagcccacgcaggctccgggtgcgcagccgagctcagcacccaagccgggggcgacgggtcgggccccgtcggtggtgaacccgaaggcgaccggggcggcgacggagcctgggacgccgtcatcgagcgcgagcccggcaccgagccggaacgcggcgccgacgccgaagccgggcatggagcccgatgagattgatcggccgtctgacggcaccatggcgcagccgaccggtgcgccagcgcgccgagtgccgcgccgacgcaggcggcgaaggccggcagcaggctgtctcgcacgggaccaacgcgctgctgatcctgggccttgcgggtgtcgcggttgtcggcgggtacctgctgctgcgggctcgccgttcgaagaactgaacacgcgacgagccggtcatccggctctgagcactgactga

SEQ ID NO: 12 - protein, 913 aa, Actinomyces viscosus
  nanH sialidase
MTSHSPFSRRRLPALLGSLPLAATGLIAAAPPAHAVPTSDGLADVTITQVNAPADGLYSVGDVMTFNITLTNTSGEAHSYAPASTNLSGNVSKCRWRNVPAGTTKTDCTGLATHTVTAEDLKAGGFTPQIAYEVKAVEYAGKALSTPETIKGATSPVKANSLRVESITPSSSQENYKLGDTVSYTVRVRSVSDKTINVAATESSFDDLGRQCHWGGLKPGKGAVYNCKPLTHTITQADVDAGRWTPSITLTATGTDGATLQTLTATGNPINVVGDHPQATPAPAPDASTELPASMSQAQHLAANTATDNYRIPAIPPPPMGTCSSPTTSARRTTATAAATTPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGPVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWCRDRGTISMSCDDGASWTTSKVFHEPFVGYTTIAVQSDGSIGLLSEDAHNGADYGGIWYRNFTMNWLGEQCGQKPAEPSPGRRRRRHPQRHRRRSRPRRPRRALSPRRHRHHPPRPSRALRPSRAGPGAGAHDRSEHGAHTGSCAQSAPEQTDGPTAAPAPETSSAPAAEPTQAPTVAPSVEPTQAPGAQPSSAPKPGATGRAPSVVNPKATGAATEPGTPSSSASPAPSRNAAPTPKPGMEPDEIDRPSDGTMAQPTGAPARRVPRRRRRRRPAAGCLARDQRAADPGPCGCRGCRRVPAAAGSPFEELNTRRAGHPALSTD

SEQ ID NO: 13 - protein, 443 aa, Artificial Sequence
  Synthetic construct
VKRKKKGGKNGKNRRNRKKKNPGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAEGADYGGIWYRNFTMNWLGEQCGQKPAE

SEQ ID NO: 14 - protein, 444 aa, Artificial Sequence
  Synthetic construct
MVKRKKKGGKNGKNRRNRKKKNPGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAEGADYGGIWYRNFTMNWLGEQCGQKPAE

(19) United States
(12) Patent Application Publication
(10) Pub. No.: US 2020/0222511 A1
(43) Pub. Date: Jul. 16, 2020

(54) TREATMENT OF MERKEL CELL POLYOMAVIRUS INFECTION

(71) Applicant: Ansun Biopharma, Inc., San Diego, CA (US)

(72) Inventor: Ronald B. Moss, Encinitas, CA (US)

(21) Appl. No.: 16/578,957

(22) Filed: Sep. 23, 2019

Related U.S. Application Data
(63) Continuation of application No. 14/894,896, filed on Nov. 30, 2015, now abandoned.
(60) Provisional application No. 61/833,414, filed on Jun. 10, 2013.

(51) Int. Cl. A61K 38/47; A61K 38/16

(52) U.S. Cl. CPC A61K 38/47; C12Y 302/01; A61K 38/164

(57) ABSTRACT

The present disclosure provides novel compositions and methods for treating an infection of the skin resulting from an infection of a member of the Orthopolyomavirus virus family. In particular, the present disclosure provides compounds having an anchoring domain that anchors the compound to the surface of a target cell, and a sialidase domain that can act extracellularly to inhibit infection of a target cell by a pathogen, such as a virus. The present disclosure also comprises therapeutic compositions having sialidase activity, including protein based compounds having sialidase catalytic domains. Compounds of the disclosure can be used for treating pathogenic infection to the skin.

### RELATED APPLICATIONS

This application is a continuation of U.S. application Ser. No. 14/894,896, filed on Nov. 30, 2015, which is the U.S. national stage under 35 USC § 371 of International Application Number PCT/US2014/041753, filed on Jun. 10, 2014, which claims priority to U.S. Application No. 61/833,414, filed on Jun. 10, 2013, the entire contents of which are hereby incorporated by reference.

### BACKGROUND

Merkel Cell Polyomavirus (MCPyV) is a member of the Polyomaviridae virus family.

Merkel cells are found in hair follicles, certain mucosal tissue and in the areas of skin involved with sensation of touch and are located in the basal level of the epidermis (Sidhu, G. S. et al. (2005) Ultrastruct. Pathol. 29: 287-294). Merkel Cell Carcinoma (MCC) is a malignancy of cutaneous neuroendocrine cells and is hypothesized to originate from the transformation of Merkel cells. It is one of the most lethal and aggressive skin cancers, and its incidence has tripled in the past 20 years and continues to climb (Hodgson, N. C. (2005) J. Surg. Oncol. 89:1-4; Lemos, B. and Nghiem, P. (2007) J. Invest. Dermatol. 127: 2100-2103.) An increase in the incidence of MCC has been noted in HIV-infected individuals (Engels, E. A. et al. (2002) Lancet 359: 497-498.), chronic lymphocytic leukemia patients (Vlad, R. and Woodlock, T. J. (2003) Am. J. Clin. Oncol. 26: 531-534.), and organ transplant patients (Buell, J. F. et al. (2002) Transplant. Proc. 34: 1780-1781.). It is prevalent in the elderly with the mean age of onset being in the 70s (Agelli, M. and Clegg, L. X. (2003) J. Am. Acad. Dermatol. 49: 832-841.) MCC also occurs almost exclusively in Caucasians at sites of the body that are frequently exposed to the sun. This link between MCC and immunosuppression led researchers to suspect an infectious origin of MCC, and MCPyV was subsequently isolated from an MCC tumor (Feng, H. et al. (2008) Science 319: 1096-1100.)

Portions of the MCPyV capsid have been shown to bind sialic acid components of the ganglioside Gtlb (Erickson, K. D. et al (2009) J. Virol. 83:10275-10279.) It has also been shown that the virus requires sulfated glycosaminoglycans, particularly heparan sulfate, for infectious entry. Although MCPyV pseudovirions efficiently bind to cells not expressing sialylated glycans like Gtlb, these particles are deficient in gene transduction. Crystal structure analysis has supported the hypothesis that the MCPyV infectious entry process requires glycosaminoglycans for initial attachment and subsequent association with sialic acid for gene transduction (Neu, U. et al (2012) PLoS Pathog. 8: e1002738.)

### SUMMARY

The present disclosure provides new compositions and methods for treating Merkel Cell Polyomavirus (MCPyV) infection and disorders associated with MCPyV infection. Specifically, it provides compounds which can act extracellularly to reduce or prevent infection of a cell by a MCPyV. Some preferred embodiments of the disclosure include therapeutic compounds having an anchoring domain that facilitates association of the compound with the surface of a target cell and a sialidase domain that can act extracellularly to reduce or prevent infection of the target cell by a pathogen, such as a virus. In some embodiments the compound comprises, consists of, or consists essentially of all or a catalytically active portion of a sialidase.

In the above method, administration of the agent having sialidase activity leads to an improvement in the parameters resulting from the infection.

Disclosed herein is a method of treating an infection by a Merkel Cell Polyomavirus (MCPyV) or MCPyV related disorder, the method comprising administering to the skin of the patient a therapeutically effective amount of an agent having sialidase activity. In various embodiments: the patient is immunocompromised; the patient is infected with HIV; the patient is suffering from chronic lymphocytic leukemia; the patient has undergone organ transplant or is being treated in preparation for organ transplant; the patient has undergone liver, heart, bone marrow or kidney transplant or is being treated in preparation for liver, heart, bone marrow or kidney transplant. In some embodiments, the agent having sialidase activity is a polypeptide comprising all or a portion of a sialidase having sialidase activity (e.g., the agent comprises a fusion protein wherein the fusion protein comprises at least a first portion comprising a portion of a sialidase having sialidase activity and a second portion that binds to a glycosaminoglycan (GAG)); or the agent comprises a fusion protein wherein the fusion protein comprises at least a first portion comprising a portion of a sialidase having sialidase activity and a second portion that has a net positive charge at physiological pH). In some cases, the second portion that binds to a GAG is selected from the group consisting of: human platelet factor 4 (SEQ ID NO: 2), human interleukin 8 (SEQ ID NO: 3), human antithrombin III (SEQ ID NO: 4), human apoprotein E (SEQ ID NO: 5), human angio associated migratory protein (SEQ ID NO: 6), and human amphiregulin (SEQ ID NO: 7). In some cases, the agent having sialidase activity is a bacterial sialidase; the bacterial sialidase is derived from a bacterium selected from the group consisting of: Vibrio cholera, Arthrobacter ureafaciens, Clostridium perfringens, Actinomyces viscosus, and Micromonospora viridifaciens. In some cases, the agent having sialidase activity is a human sialidase. The agent can be topically administered to the skin; e.g., the infected skin or the skin most frequently exposed to the sun. The agent can be administered by subdermal injection, or as a lotion or a transdermal patch. The administration of the agent having sialidase activity causes one or more of: a decrease in malignant lesions on the skin, and a reduction of MCPyV viral load. In some cases, the infection of the skin is associated with an event selected from the group consisting of an HIV infection and commencement of immunosuppressive therapy.

In some cases the agent having sialidase activity comprises, consists of, or consists essentially of DAS181 (SEQ ID NO:13 or SEQ ID NO:14). In some cases, the method comprises administering a topical composition comprising microparticles comprising a compound that comprises, consists of, or consists essentially of DAS181 (SEQ ID NO:13 or SEQ ID NO:14).

In some cases, the agent having sialidase activity is DAS181. In some cases the method comprises administering composition comprising microparticles comprising DAS181.

### DETAILED DESCRIPTION

In general, the present disclosure relates to methods for treating MCPyV virus infection using agents having sialidase activity. Suitable agents are described in U.S. Pat. Nos. 8,084,036 and 7,807,174 which are both hereby incorporated by reference in their entirety. The agents having sialidase activity can remove sialic acid residues from the surface of cells and reduce in infection by certain viruses that binding to sialic acid residues, e.g., MCPyV.

In some embodiments, the severity of the infection is reduced with the treatment of the compounds. The reduction of the severity of the infection can be measured by the reduction of the symptoms which present with the infection.

The compounds of the present disclosure have sialidase activity. In some instances, the compounds having sialidase activity are a fusion protein in which the portion having sialidase activity is fused to a protein or protein fragment not having sialidase activity. In some instances the portion having sialidase activity is fused to an anchoring domain. In some instances the anchoring domain is GAG.

DAS181 (SEQ ID NOs: 13 and 14) is a fusion protein compound comprising the catalytic domain of a sialidase (A. viscous) and an anchoring domain that is a human amphiregulin GAG-binding domain. In some instances of the present disclosure, DAS181 could be used to treat the infection by MCPyV virus and disorders associated therewith (e.g., Merkel Cell Carcinoma).

Unless defined otherwise, all technical and scientific terms used herein have the same meaning as commonly understood by one of ordinary skill in the art to which this disclosure belongs. Generally, the nomenclature used herein and the manufacture or laboratory procedures described below are well known and commonly employed in the art. Conventional methods are used for these procedures, such as those provided in the art and various general references. Where a term is provided in the singular, the inventors also contemplate the plural of that term. Where there are discrepancies in terms and definitions used in references that are incorporated by reference, the terms used in this application shall have the definitions given herein. As employed throughout the disclosure, the following terms, unless otherwise indicated, shall be understood to have the following meanings:

A “target cell” is any cell that can be infected by MCPyV virus, such as a merkel cell.

A “domain that can anchor said at least one sialidase domain to the membrane of a target cell”, also called an “extracellular anchoring domain” or simply, “anchoring domain” refers to a moiety that can interact with a moiety that is at or on the exterior of a cell surface or is in close proximity to the surface of a cell. An extracellular anchoring domain can be reversibly or irreversibly linked to one or more moieties, such as, preferably, one or more sialidase domains, and thereby cause the one or more attached therapeutic moieties to be retained at or in close proximity to the exterior surface of a eukaryotic cell. Preferably, an extracellular anchoring domain interacts with at least one molecule on the surface of a target cell or at least one molecule found in close association with the surface of a target cell. For example, an extracellular anchoring domain can bind a molecule covalently or noncovalently associated with the cell membrane of a target cell, or can bind a molecule present in the extracellular matrix surrounding a target cell. An extracellular anchoring domain preferably is a peptide, polypeptide, or protein, and can also comprise any additional type of chemical entity, including one or more additional proteins, polypeptides, or peptides, a nucleic acid, peptide nucleic acid, nucleic acid analogue, nucleotide, nucleotide analogue, small organic molecule, polymer, lipids, steroid, fatty acid, carbohydrate, or a combination of any of these.

As used herein, a protein or peptide sequences is “substantially homologous” to a reference sequence when it is either identical to a reference sequence, or comprises one or more amino acid deletions, one or more additional amino acids, or more one or more conservative amino acid substitutions, and retains the same or essentially the same activity as the reference sequence. Conservative substitutions may be defined as exchanges within one of the following five groups:

I. Small, aliphatic, nonpolar or slightly polar residues: Ala, Ser, Thr, Pro, Gly

II. Polar, negatively charged residues and their amides: Asp, Asn, Glu, Gln

III. Polar, positively charged residues: His, Arg, Lys

IV. Large, aliphatic nonpolar residues: Met, Leu, Ile, Val, Cys

V. Large aromatic residues: Phe, Try, Trp

Within the foregoing groups, the following substitution are considered to be “highly conservative”: Asp/Glu, His/Arg/Lys, Phe/Tyr/Trp, and Met/Leu/Ile/Val. Semi-conservative substitutions are defined to be exchanges between two of groups (I)-(V) above which are limited to supergroup (A), comprising (I), (II), and (III) above, or to supergroup (B), comprising (IV) and (V) above. In addition, where hydrophobic amino acids are specified in the application, they refer to the amino acids Ala, Gly, Pro, Met, Leu, Ile, Val, Cys, Phe, and Trp, whereas hydrophilic amino acids refer to Ser, Thr, Asp, Asn, Glu, Gln, His, Arg, Lys, and Tyr.

As used herein, the phrase “therapeutically effective amount” refers to the amounts of active compounds or their combination that elicit the biological or medicinal response that is being sought in a tissue, system, animal, individual or human by a researcher, veterinarian, medical doctor or other clinician, which includes one or more of the following:

(1) inhibiting the disease and its progression; for example, inhibiting a disease, condition or disorder in an individual who is experiencing or displaying the pathology or symptomatology of the disease, condition or disorder (i.e., arresting further development of the pathology and/or symptomatology) such as in the case of MCPyV virus infection, and

(2) ameliorating the disease; for example, ameliorating a disease, condition or disorder in an individual who is experiencing or displaying the pathology or symptomatology of the disease, condition or disorder (i.e., reversing the pathology and/or symptomatology) such as in the case of MCPyV virus infection.

As used herein, the phrase “treating (including treatment)” includes one or more of the following:

(1) inhibiting the disease and its progression; for example, inhibiting a disease, condition or disorder in an individual who is experiencing or displaying the pathology or symptomatology of the disease, condition or disorder (i.e., arresting further development of the pathology and/or symptomatology), and

(2) ameliorating the disease; for example, ameliorating a disease, condition or disorder in an individual who is experiencing or displaying the pathology or symptomatology of the disease, condition or disorder.

A “sialidase” is an enzyme that can remove a sialic acid residue from a substrate molecule. The sialidases (N-acylneuraminosylglycohydrolases, EC 3.2.1.18) are a group of enzymes that hydrolytically remove sialic acid residues from sialo-glycoconjugates. Sialic acids are alpha-keto acids with 9-carbon backbones that are usually found at the outermost positions of the oligosaccharide chains that are attached to glycoproteins and glycolipids. One of the major types of sialic acids is N-acetylneuraminic acid (NeuSAc), which is the biosynthetic precursor for most of the other types. The substrate molecule can be, as nonlimiting examples, an oligosaccharide, a polysaccharide, a glycoprotein, a ganglioside, or a synthetic molecule. For example, a sialidase can cleave bonds having alpha (2,3)-Gal, alpha(2,6)-Gal, or alpha (2,8)-Gal linkages between a sialic acid residue and the remainder of a substrate molecule. A sialidase can also cleave any or all of the linkages between the sialic acid residue and the remainder of the substrate molecule. Two major linkages between NeuSAc and the penultimate galactose residues of carbohydrate side chains are found in nature, NeuSAc alpha (2,3)-Gal and NeuSAc alpha (2,6)-Gal. Both NeuSAc alpha (2,3)-Gal and NeuSAc alpha (2,6)-Gal molecules can be recognized by influenza viruses as the receptor, although human viruses seem to prefer NeuSAc alpha (2,6)-Gal, avian and equine viruses predominantly recognize NeuSAc alpha (2,3)Gal. A sialidase can be a naturally-occurring sialidase, an engineered sialidase (such as, but not limited to a sialidase whose amino acid sequence is based on the sequence of a naturally-occurring sialidase, including a sequence that is substantially homologous to the sequence of a naturally-occurring sialidase). As used herein, “sialidase” can also mean the active portion of a naturally-occurring sialidase, or a peptide or protein that comprises sequences based on the active portion of a naturally-occurring sialidase.

A “fusion protein” is a protein comprising amino acid sequences from at least two different sources. A fusion protein can comprise amino acid sequence that is derived from a naturally occurring protein or is substantially homologous to all or a portion of a naturally occurring protein, and in addition can comprise from one to a very large number of amino acids that are derived from or substantially homologous to all or a portion of a different naturally occurring protein. In the alternative, a fusion protein can comprise amino acid sequence that is derived from a naturally occurring protein or is substantially homologous to all or a portion of a naturally occurring protein, and in addition can comprise from one to a very large number of amino acids that are synthetic sequences.

A “sialidase catalytic domain protein” is a protein that comprises the catalytic domain of a sialidase, or an amino acid sequence that is substantially homologous to the catalytic domain of a sialidase, but does not comprises the entire amino acid sequence of the sialidase the catalytic domain is derived from, wherein the sialidase catalytic domain protein retains substantially the same activity as the intact sialidase the catalytic domain is derived from. A sialidase catalytic domain protein can comprise amino acid sequences that are not derived from a sialidase, but this is not required. A sialidase catalytic domain protein can comprise amino acid sequences that are derived from or substantially homologous to amino acid sequences of one or more other known proteins, or can comprise one or more amino acids that are not derived from or substantially homologous to amino acid sequences of other known proteins.

### I. Composition for Preventing or Treating Infection by a Pathogen

The present disclosure relates to compounds (agents) that include a peptide. The compounds include all or a catalytic portion of a sialidase. In some cases the compound includes at least one domain that can associate the sialidase or portion thereof with a eukaryotic cell. By “peptide or protein-based” compounds, it is meant that a compound that includes a portion having an amino acid framework, in which the amino acids are joined by peptide bonds. A peptide or protein-based compound can also have other chemical compounds or groups attached to the amino acid framework or backbone, including moieties that contribute to the anchoring activity of the anchoring domain, or moieties that contribute to the infection-preventing activity or the sialidase domain. For example, the protein-based therapeutics of the present disclosure can comprise compounds and molecules such as but not limited to: carbohydrates, fatty acids, lipids, steroids, nucleotides, nucleotide analogues, nucleic acid molecules, nucleic acid analogues, peptide nucleic acid molecules, small organic molecules, or even polymers. The protein-based therapeutics of the present disclosure can also comprise modified or non-naturally occurring amino acids. Non-amino acid portions of the compounds can serve any purpose, including but not limited to: facilitating the purification of the compound, improving the solubility or distribution or the compound (such as in a therapeutic formulation), linking domains of the compound or linking chemical moieties to the compound, contributing to the two dimensional or three-dimensional structure of the compound, increasing the overall size of the compound, increasing the stability of the compound, and contributing to the anchoring activity or therapeutic activity of the compound.

The peptide or protein-based compounds of the present disclosure can also include protein or peptide sequences in addition to those that comprise anchoring domains or sialidase domains. The additional protein sequences can serve any purpose, including but not limited to any of the purposes outlined above (facilitating the purification of the compound, improving the solubility or distribution or the compound, linking domains of the compound or linking chemical moieties to the compound, contributing to the two-dimensional or three-dimensional structure of the compound, increasing the overall size of the compound, increasing the stability of the compound, or contributing to the anchoring activity or therapeutic activity of the compound). Preferably any additional protein or amino acid sequences are part of a single polypeptide or protein chain that includes the sialidase domain or domains, but any feasible arrangement of protein sequences is within the scope of the present disclosure.

The anchoring domain and sialidase domain can be arranged in any appropriate way that allows the compound to bind at or near a target cell membrane such that the therapeutic sialidase can exhibit an extracellular activity that prevents or impedes infection of the target cell by a pathogen. The compound will preferably have at least one protein or peptide-based anchoring domain and at least one peptide or protein-based sialidase domain. In this case, the domains can be arranged linearly along the peptide backbone in any order. The anchoring domain can be N-terminal to the sialidase domain, or can be C-terminal to the sialidase domain.

It is also possible to have one or more sialidase domains flanked by at least one anchoring domain on each end. Alternatively, one or more anchoring domains can be flanked by at least one sialidase domain on each end. Chemical, or preferably, peptide, linkers can optionally be used to join some or all of the domains of a compound. It is also possible to have the domains in a nonlinear, branched arrangement. For example, the sialidase domain can be attached to a derivatized side chain of an amino acid that is part of a polypeptide chain that also includes, or is linked to, the anchoring domain.

A compound of the present disclosure can have more than one anchoring domain. In cases in which a compound has more than one anchoring domain, the anchoring domains can be the same or different. A compound of the present disclosure can have more than one sialidase domain. In cases in which a compound has more than one sialidase domain, the sialidase domains can be the same or different. Where a compound comprises multiple anchoring domains, the anchoring domains can be arranged in tandem (with or without linkers) or on alternate sides of other domains, such as sialidase domains. Where a compound comprises multiple sialidase domains, the sialidase domains can be arranged in tandem (with or without linkers) or on alternate sides of other domains, such as, but not limited to, anchoring domains.

A peptide or protein-based compound of the present disclosure can be made by any appropriate way, including purifying naturally occurring proteins, optionally proteolytically cleaving the proteins to obtain the desired functional domains, and conjugating the functional domains to other functional domains. Peptides can also be chemically synthesized, and optionally chemically conjugated to other peptides or chemical moieties. Preferably, however, a peptide or protein-based compound of the present disclosure is made by engineering a nucleic acid construct to encode at least one anchoring domain and at least one sialidase domain together (with or without nucleic acid linkers) in a continuous polypeptide. The nucleic acid constructs, preferably having appropriate expression sequences, can be transfected into prokaryotic or eukaryotic cells, and the therapeutic protein-based compound can be expressed by the cells and purified. Any desired chemical moieties can optionally be conjugated to the peptide or protein-based compound after purification. In some cases, cell lines can be chosen for expressing the protein-based therapeutic for their ability to perform desirable post-translational modifications (such as, but not limited to glycosylation).

A great variety of constructs can be designed and their protein products tested for desirable activities (such as, for example, binding activity of an anchoring domain or catalytic activity of a sialidase domain). The protein products of nucleic acid constructs can also be tested for their efficacy in preventing or impeding infection of a target cell by a pathogen. In vitro and in vivo tests for the infectivity of pathogens are known in the art.

### Anchoring Domain

As used herein, an “extracellular anchoring domain” or “anchoring domain” is any moiety that interact with an entity that is at or on the exterior surface of a target cell or is in close proximity to the exterior surface of a target cell. An anchoring domain serves to retain a compound of the present disclosure at or near the external surface of a target cell. An extracellular anchoring domain preferably binds 1) a molecule expressed on the surface of a target cell, or a moiety, domain, or epitope of a molecule expressed on the surface of a target cell, 2) a chemical entity attached to a molecule expressed on the surface of a target cell, or 3) a molecule of the extracellular matrix surrounding a target cell.

An anchoring domain is preferably a peptide or protein domain (including a modified or derivatized peptide or protein domain), or comprises a moiety coupled to a peptide or protein. A moiety coupled to a peptide or protein can be any type of molecule that can contribute to the interaction of the anchoring domain to an entity at or near the target cell surface, and is preferably an organic molecule, such as, for example, nucleic acid, peptide nucleic acid, nucleic acid analogue, nucleotide, nucleotide analogue, small organic molecule, polymer, lipids, steroid, fatty acid, carbohydrate, or any combination of any of these.

Target tissue or target cell type includes the sites in an animal or human body where a pathogen invades or amplifies. For example, a target cell can be a kidney cell that can be infected by a MCPyV virus. A compound or agents of the present disclosure can comprise an anchoring domain that can interact with a cell surface entity, for example, that is specific for the target cell type.

A compound for treating infection by a pathogen can comprise an anchoring domain that can bind at or near the surface of a target cell. For example, heparin/sulfate, closely related to heparin, is a type of GAG that is ubiquitously present on cell membranes, including the surface of respiratory epithelium. Many proteins specifically bind to heparin/heparan sulfate, and the GAG-binding sequences in these proteins have been identified (Meyer, F A, King, M and Gelman, R A. (1975) Biochimica et BiophysicaActa 392: 223-232; Schauer, S. ed., pp 233. Sialic Acids Chemistry, Metabolism and Function. Springer-Verlag, 1982). For example, the GAG-binding sequences of human platelet factor 4 (PF4) (SEQ ID NO:2), human interleukin 8 (IL8) (SEQ ID NO:3), humanantithrombin III (AT III) (SEQ ID NO:4), human apoprotein E (ApoE) (SEQ ID NO:5), human angio-associated migratory cell protein (AAMP) (SEQ ID NO:6), or human amphiregulin (SEQ ID NO:7) have been shown to have very high affinity (in the nanomolar range) towards heparin (Lee, M K and Lander, A D. (1991) Pro Natl Acad Sci USA 88:2768-2772; Goger, B, Halden, Y, Rek, A, Mosl, R, Pye, D. Gallagher, J and Kungl, A J. (2002) Biochem. 41:1640-1646; Witt, D P and Lander A D (1994) Curr Bio 4:394-400; Weisgraber, K H, Rail, S C, Mahley, R W, Milne, R W and Marcel, Y. (1986) J Bio Chern 261:2068-2076). These sequences, or other sequences that have been identified or are identified in the future as heparin/heparan sulfate binding sequences, or sequences substantially homologous to identified heparin/heparan sulfate binding sequences that have heparin/heparan sulfate binding activity, can be used as epithelium-anchoring-domains in compounds of the present disclosure that can be used.

### Sialidase Domain

A sialidase that can cleave more than one type of linkage between a sialic acid residue and the remainder of a substrate molecule, in particular, a sialidase that can cleave both α(2,6)-Gal and α(2,3)-Gal linkages can be used in the compounds of the disclosure. Sialidases include are the large bacterial sialidases that can degrade the receptor sialic acids Neu5Ac alpha(2,6)-Gal and Neu5Ac alpha(2,3)-Gal. For example, the bacterial sialidase enzymes from Clostridium perfringens (Genbank Accession Number X87369), Actinomyces viscosus, Arthrobacter ureafaciens, or Micromonospora viridifaciens (Genbank Accession Number D01045) can be used. Sialidase domains of compounds of the present disclosure can comprise all or a portion of the amino acid sequence of a large bacterial sialidase or can comprise amino acid sequences that are substantially homologous to all or a portion of the amino acid sequence of a large bacterial sialidase. In one preferred embodiment, a sialidase domain comprises a sialidase encoded by Actinomyces viscosus, such as that of SEQ ID NO: 12, or such as sialidase sequence substantially homologous to SEQ ID NO: 12. In yet another preferred embodiment, a sialidase domain comprises the catalytic domain of the Actinomyces viscosus sialidase extending from amino acids 274-666 of SEQ ID NO:12, or a substantially homologous sequence.

Additional sialidases include the human sialidases such as those encoded by the genes NEU2 (SEQ ID NO:8; Genbank Accession Number Y16535; Monti, E, Preti, Rossi, E., Ballabio, A and Borsani G. (1999) Genomics 57:137-143) and NEU4 (SEQ ID NO:9; Genbank Accession Number NM080741; Monti, E, Preti, A, Venerando, Band Borsani, G. (2002) Neurochem Res 27:646-663). Sialidase domains of compounds of the present disclosure can comprise all or a portion of the amino acid sequences of a sialidase or can comprise amino acid sequences that are substantially homologous to all or a portion of the amino acid sequences of a sialidase. Preferably, where a sialidase domain comprises a portion of the amino acid sequences of a naturally occurring sialidase, or sequences substantially homologous to a portion of the amino acid sequences of a naturally occurring sialidase, the portion comprises essentially the same activity as the intact sialidase. The present disclosure also includes sialidase catalytic domain proteins. As used herein a “sialidase catalytic domain protein” comprises a catalytic domain of a sialidase but does not comprise the entire amino acid sequence of the sialidase from which the catalytic domain is derived. A sialidase catalytic domain protein has sialidase activity. Preferably, a sialidase catalytic domain protein comprises at least 10%, at least 20%, at least 50%, at least 70% of the activity of the sialidase from which the catalytic domain sequence is derived. More preferably, a sialidase catalytic domain protein comprises at least 90% of the activity of the sialidase from which the catalytic domain sequence is derived.

A sialidase catalytic domain protein can include other amino acid sequences, such as but not limited to additional sialidase sequences, sequences derived from other proteins, or sequences that are not derived from sequences of naturally occurring proteins. Additional amino acid sequences can perform any of a number of functions, including contributing other activities to the catalytic domain protein, enhancing the expression, processing, folding, or stability of the sialidase catalytic domain protein, or even providing a desirable size or spacing of the protein.

A preferred sialidase catalytic domain protein is a protein that comprises the catalytic domain of the A. viscosus sialidase. Preferably, an A. viscosus sialidase catalytic domain protein comprises amino acids 270-666 of the A. viscosus sialidase sequence (SEQ ID NO:12). Preferably, an A. viscosus sialidase catalytic domain protein comprises an amino acid sequence that begins at any of the amino acids from amino acid 270 to amino acid 290 of the A. viscosus sialidase sequence (SEQ ID NO: 12) and ends at any of the amino acids from amino acid 665 to amino acid 901 of said A. viscosus sialidase sequence (SEQ ID NO: 12), and lacks any A. viscosus sialidase protein sequence extending from amino acid 1 to amino acid 269. (As used herein “lacks any A. viscosus sialidase protein sequence extending from amino acid 1 to amino acid 269” means lacks any stretch of four or more consecutive amino acids as they appear in the designated protein or amino acid sequence.)

In some preferred embodiments, an A. viscosus sialidase catalytic domain protein comprises amino acids 274-681 of the A. viscosus sialidase sequence (SEQ ID NO: 12) and lacks other A. viscosus sialidase sequence. In some preferred embodiments, an A. viscosus sialidase catalytic domain protein comprises amino acids 274-666 of the A. viscosus sialidase sequence (SEQ ID NO: 12) and lacks any other A. viscosus sialidase sequence. In some preferred embodiments, an A. viscosus sialidase catalytic domain protein comprises amino acids 290-666 of the A. viscosus sialidase sequence (SEQ ID NO: 12) and lacks any other A. viscosus sialidase sequence. In yet other preferred embodiments, an A. viscosus sialidase catalytic domain protein comprises amino acids 290-681 of the A. viscosus sialidase sequence (SEQ ID NO: 12) and lacks any other A. viscosus sialidase sequence.

### Linkers

A compound of the present disclosure can optionally include one or more linkers that can join domains of the compound. Linkers can be used to provide optimal spacing or folding of the domains of a compound. The domains of a compound joined by linkers can be sialidase domains, anchoring domains, or any other domains or moieties of the compound that provide additional functions such as enhancing compound stability, facilitating purification, etc. A linker used to join domains of compounds of the present disclosure can be a chemical linker or an amino acid or peptide linker. Where a compound comprises more than one linker, the linkers can be the same or different. Where a compound comprises more than one linker, the linkers can be of the same or different lengths.

Many chemical linkers of various compositions, polarity, reactivity, length, flexibility, and cleavability are known in the art of organic chemistry. Preferred linkers of the present disclosure include amino acid or peptide linkers. Peptide linkers are well known in the art. Preferably linkers are between one and one hundred amino acids in length, and more preferably between one and thirty amino acids in length, although length is not a limitation in the linkers of the compounds of the present disclosure. Preferably linkers comprise amino acid sequences that do not interfere with the conformation and activity of peptides or proteins encoded by monomers of the present disclosure. Some preferred linkers of the present disclosure are those that include the amino acid glycine. For example, linkers having the sequence: (GGGGS (SEQ ID NO:10))n, where n is a whole number between I and 20, or more preferably between I and 12, can be used to link domains of therapeutic compounds of the present disclosure.

The present disclosure also includes nucleic acid molecules that encode protein-based compounds of the present disclosure that comprise at least one sialidase domain and at least one anchoring domain. The nucleic acid molecules can have codons optimized for expression in particular cell types, such as, for example E. coli or human cells. The nucleic acid molecules or the present disclosure that encode protein-based compounds of the present disclosure that comprise at least one sialidase domain and at least one anchoring domain can also comprise other nucleic acid sequences, including but not limited to sequences that enhance gene expression. The nucleic acid molecules can be in vectors, such as but not limited to expression vectors.

### Administration

The compound is administered so that it comes into contact with the target cells, but is preferably not administered systemically to the patient. Thus, a composition comprising a sialidase (e.g., a composition comprising DAS181) can be topically administered to the skin, e.g., the infected skin or the skin most frequently exposed to the sun. The composition can be administered by subdermal injection, or as a lotion or a transdermal patch to the infected skin. The administration of the agent having sialidase activity causes one or more of: a decrease in malignant lesions on the skin, and a reduction of MCPyV viral load.

### Nucleic Acid Molecules

The present disclosure also comprises nucleic acid molecules that encode protein-based compounds of the present disclosure that comprise a catalytic domain of a sialidase. The nucleic acid molecules can have codons optimized for expression in particular cell types, such as, for example E. coli or human cells. The nucleic acid molecules or the present disclosure that encode protein-based compounds of the present disclosure that comprise at least one catalytic domain of a sialidase can also comprise other nucleic acid sequences, including but not limited to sequences that enhance gene expression. The nucleic acid molecules can be in vectors, such as but not limited to expression vectors.

### II. Pharmaceutical Compositions

The present disclosure includes compounds of the present disclosure formulated as pharmaceutical compositions. The pharmaceutical compositions comprise a pharmaceutically acceptable carrier prepared for storage and preferably subsequent administration, which have a pharmaceutically effective amount of the compound in a pharmaceutically acceptable carrier or diluent. Acceptable carriers or diluents for therapeutic use are well known in the pharmaceutical art, and are described, for example, in Remington's Pharmaceutical Sciences, 18th Ed., Mack Publishing Co., Easton, Pa. (1990)). Preservatives, stabilizers, dyes and even flavoring agents can be provided in the pharmaceutical composition. For example, sodium benzoate, sorbic acid and esters of p-hydroxybenzoic acid can be added as preservatives. In addition, antioxidants and suspending agents can be used.

The pharmaceutically effective amount of a test compound required as a dose will depend on the route of administration, the type of animal or patient being treated, and the physical characteristics of the specific animal under consideration. The dose can be tailored to achieve a desired effect, but will depend on such factors as weight, diet, concurrent medication and other factors which those skilled in the medical arts will recognize. In practicing the methods of the present disclosure, the pharmaceutical compositions can be used alone or in combination with one another, or in combination with other therapeutic or diagnostic agents. These products can be utilized in vivo, preferably in a mammalian patient, preferably in a human, or in vitro. In employing them in vivo, the pharmaceutical compositions can be administered to the patient in a variety of ways, preferably topically to the target cells, topically to the locus of infection or topically to tissue comprising the target cells.

Accordingly, in some embodiments, the methods comprise administration of the agent and a pharmaceutically acceptable carrier. In some embodiments, the ophthalmic composition is a liquid composition, semi-solid composition, insert, film, microparticles or nanoparticles.

### III. Method of Treating an Infection by a Pathogen

The method of the present disclosure includes: treating a subject that is infected with a pathogen or at risk of being infected with a pathogen with a pharmaceutical composition of the present disclosure that comprises a protein-based compound that comprises a sialidase activity. In some preferred embodiments the method includes applying a therapeutically effective amount of a pharmaceutical composition of the present disclosure to target cells of a subject. The sialidase activity can be an isolated naturally occurring sialidase protein, or a recombinant protein substantially homologous to at least a portion of a naturally occurring sialidase. A preferred pharmaceutical composition comprises a sialidase with substantial homology to the A. viscosus sialidase (SEQ ID NO:12). The subject to be treated can be an animal or human subject. In yet another aspect, the method includes: treating a subject that is infected with a pathogen with a pharmaceutical composition of the present disclosure that comprises a protein-based compound that comprises a sialidase catalytic domain. In some preferred embodiments, the method includes applying a therapeutically effective amount of a pharmaceutical composition of the present disclosure to epithelial cells of a subject. The sialidase catalytic domain is preferably substantially homologous to the catalytic domain of a naturally occurring sialidase. A preferred pharmaceutical composition comprises a sialidase catalytic domain with substantial homology to amino acids 274-666 the A. viscosus sialidase (SEQ ID NO: 12). The subject to be treated can be an animal or human subject. In some cases the compound is DAS181.

### Dosage

As will be readily apparent to one skilled in the art, the useful in vivo dosage to be administered and the particular mode of administration will vary depending upon the age, weight and type of patient being treated, the particular pharmaceutical composition employed, and the specific use for which the pharmaceutical composition is employed. The determination of effective dosage levels, that is the dose levels necessary to achieve the desired result, can be accomplished by one skilled in the art using routine methods as discussed above. In non-human animal studies, applications of the pharmaceutical compositions are commenced at higher dose levels, with the dosage being decreased until the desired effect is no longer achieved or adverse side effects are reduced or disappear. The dosage for a compound of the present disclosure can range broadly depending upon the desired affects, the therapeutic indication, route of administration and purity and activity of the compound. Typically, human clinical applications of products are commenced at lower dosage levels, with dosage level being increased until the desired effect is achieved. Alternatively, acceptable in vitro studies can be used to establish useful doses and routes of administration of the test compound. Typically, dosages can be between about 1 ng/kg and about 10 mg/kg, preferably between about 10 ng/kg and about 1 mg/kg, and more preferably between about 100 ng/kg and about 100 micrograms/kg.

In one preferred regimen, appropriate dosages are administered to each patient by subdermal injection, or as a lotion or a transdermal patch to the infected skin. It will be understood, however, that the specific dose level and frequency of dosage for any particular patient maybe varied and will depend upon a variety of factors including the activity of the specific salt or other form employed, the metabolic stability and length of action of that compound, the age of the patient, body weight of the patient, general health of the patient, sex of the patient, diet of the patient, mode and time of administration, rate of excretion, drug combination, the severity of the particular condition, and the host undergoing therapy.

### EXAMPLES

### Example 1: Preparation of a Suspension of DAS181 Microparticles for Use in Treating Infections

### Purification of DAS181

DAS181 is a fusion protein containing the heparin (glycosaminoglycan, or GAG) binding domain from human amphiregulin fused via its N-terminus to the C-terminus of a catalytic domain of Actinomyces viscosus (e.g., sequence of amino acids set forth in SEQ ID NO: 13 (no amino terminal methionine) and SEQ ID NO: 14 (including amino terminal methionine). The DAS181 protein used in the examples below was purified as described in Malakhov et al., Antimicrob. Agents Chemother., 1470-1479 (2006), which is incorporated in its entirety by reference herein. Briefly, the DNA fragment coding for DAS181 was cloned into the plasmid vector pTrc99a (Pharmacia) under the control of an IPTG (isopropyl-β-D-thiogalactopyranoside)-inducible promoter. The resulting construct was expressed in the BL21 strain of Escherichia coli (E. coli). The E. coli cells expressing the DAS181 protein were washed by diafiltration in a fermentation harvest wash step using Toyopearl buffer 1, UFP-500-E55 hollow fiber cartridge (GE Healthcare) and a Watson-Marlow peristaltic pump. The recombinant DAS181 protein was then purified in bulk from the cells as described in US 20050004020 and US 20080075708, which are incorporated in their entirety by reference herein.

### Activity of DAS181

The sialidase activity of DAS181 was measured using the fluorogenic substrate 4-methylumbelliferyl-N-acetyl-α-D-neuraminic acid (4-MU-NANA; Sigma). One unit of sialidase is defined as the amount of enzyme that releases 10 nmol of MU from 4-MU-NANA in 10 minutes at 37° C. (50 mM CH₃COOH—NaOH buffer, pH 5.5) in a reaction that contains 20 nmol of 4-MU-NANA in a 0.2 ml volume (Potier et al., Anal. Biochem., 94:287-296, 1979). The specific activity of DAS181 was determined to be 1,300 U/mg protein (0.77 μg DAS181 protein per unit of activity).

### Microparticle Preparation

The following ingredients were then combined to form DAS181 microparticles in a large scale batch process:
(a) 75 mg/ml Histidine, 0.107M citric acid, pH 5.0 and 1M Trehalose stock solutions were sterile filtered into and combined in an Excipient Bottle.
(b) The contents of the Excipient Bottle were added, with mixing, to a Compounding Vessel containing 125 mg/ml DAS181 protein prepared as described in Example 1.
(c) Isopropanol was sterile filtered into an Isopropanol Bag
(d) The content of the Isopropanol Bag was pumped into the Compounding Vessel while mixing vigorously to form the Feedstock Solution. The final composition of the Feedstock Solution was as follows: 70 mg/ml DAS181, 26% isopropanol, 9.8 mg/ml histidine, 9.8 mg/ml trehalose, 2.69 mg/ml citric acid, pH 5.0. The time between initiating the addition of isopropanol and starting the lyophilization cycle was between 90 minutes and 120 minutes
(e) Stainless Steel trays that had undergone depyrogenation were each filled with 950 g of the Feedstock Solution, using a metering pump
(f) The filled Stainless Steel trays were subjected to a Lyophilization Cycle as follows:
   a. the feedstock solution in the lyophilization trays were gasketed and placed in the lyophilizer shelves at 25° C. for 5 minutes;
   b. the temperature of the shelves was lowered to −55° C. at a ramp rate of −0.4° C./minute;
   c. the trays were held at −55° C. for between 60 and 180 minutes;
   d. primary drying was accomplished by setting the condenser to <−60° C., applying a vacuum of 125 mTorr with 250 mTorr dead band and increasing the temperature to −40° C. at a ramp rate of 0.125° C./minute and further to a temperature of −30° C. at 0.167° C./minute;
   e. the temperature was held at −30° C. for between 5000 and 6500 minutes;
   f. secondary drying was accomplished by increasing the temperature to 15° C. at a ramp rate of 0.5° C./minute, holding at 15° C. for 30 minutes, then further ramping up to a temperature of 30° C. at a ramp rate of 0.5° C./minute;
   g. the temperature was held at 30° C. for between 300 and 500 minutes; and
   h. the vacuum was released and the lyophilizer was backfilled with nitrogen to prevent oxidation of the microparticle formulations before transferring into bottles for bulk mixing and aliquoting the bulk powder for storage at ≤−15° C.

### Physical Parameters

The DAS181 dry powder microparticles prepared according to the above method have a mass median aerodynamic diameter (MMAD) of about 10 microns and a GSD of between 1 and 2.

### Suspension of Microparticles

To prepare 1 ml of a 100 mg DAS181/ml suspension, 125 mg of microparticles prepared as described were placed in a vial in a controlled RH environment (typically 10-30% RH). Next, 450 μL of PEG 300 was added to the vial and gently mixed with the microparticles. The mixture was held for 5 minutes to allow the microparticles to interact with the PEG 300. Next, 450 μL of water is added to the vial and the contents are gently mixed for 2-3 minutes or until a homogeneous suspension is achieved.

Injectability was measured using a NE-1010 syringe pump with a DPM-3 digital mount meter attached to the plunger rail. Standard 1 mL BD syringes are used with 27 G×½ PrecisionGlide BD needles. Injectability values are reported in unit of lbs of force measured. Viscosity was measured using a Brookfield DV-1 Prime with a CPE-44PY cup and a CPE-40 cone spindle. Injection force of less than 50N is considered as injectable. The conversion unit of lbs to N is 1 lbs=4.4 N.

The above method produced suspensions with good injectability. Good results were obtained when the ratio of PEG 300 to water was: 50:50, 65:35 and 75:25. When PEG 200 was used, good results were obtained when the ratio of PEG 300 to water was 65:35 and 75:25.

In addition to polyethylene glycol (PEG 200, PEG 300, PEG 400, PEG 500, PEG 600), polysorbate 80, polysorbate 20 (Polyoxyethylene (20) sorbitan monooleate), propylene glycol, thioglycerol, tricaprylin, triolein, and versetamide are useful first media for adding to the protein microparticles.

The second media is water that can include salts, buffers, preservatives and other pharmaceutically acceptable excipients.

1. A method of treating an infection by a Merkel Cell Polyomavirus (MCPyV) or MCPyV-related disorder, the method comprising administering to the skin of a patient an effective amount of an agent having sialidase activity.

2.-24. (canceled)
